(19) United States
(12) Patent Application Publication
(10) Pub. No.: US 2008/0268056 A1
(43) Pub. Date: Oct. 30, 2008

(54) INJECTABLE COPOLYMER HYDROGEL USEFUL FOR REPAIRING VERTEBRAL COMPRESSION FRACTURES

(76) Inventor: ABHIJEET JOSHI, Austin, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/740,588

(22) Filed: Apr. 26, 2007

(51) Int. Cl. A61K 9/00; A61K 47/30; A61P 19/00

(52) U.S. Cl. 424/486; 514/772.3

(57) ABSTRACT

Embodiments of the present invention provide a biocompatible substance useful for repairing a vertebral compression fracture (VCF). The biocompatible substance can be made from two or more biocompatible polymeric hydrogels via physical cross-linking. The biocompatible substance thus made exhibits a lower critical solution temperature (LCST) phenomenon and undergoes volume and stage changes with temperature in the range of 25° C.-34° C. The biocompatible substance can be in liquid injectable form at room temperature, can cure within the human body without damaging living tissues, does not release toxic monomers during surgery, and, once cured, has optimum mechanical properties that match those of a human cancellous bone. It can be used alone in minimally invasive procedures or in combination with other spine treatments.

### FIELD OF THE INVENTION

This invention relates generally to implants. More particularly, the present invention relates to biocompatible implants useful in repairing vertebral compression fractures. Even more particularly, the present invention relates to a biocompatible substance which can be injected in liquid form in the vicinity of a vertebral compression fracture and cured in the human body without harmful side effects.

### BACKGROUND OF THE INVENTION

In healthy people, the bones of the spine are generally strong In people who have suffered a trauma (e.g., injury from a big fall or car accident) and who have a certain disease (e.g., osteoporosis, cancer, degenerative bone disease, etc.), these same bones can break or fracture Compression fracture of the vertebral bodies is particularly common in patients with osteoporosis and is an especially frequent phenomenon in elderly people. Due to the reduced bone density, vertebral compression fractures (VCF) may occur with little or no trauma The prevalence of this condition increases with advancing age.

The vertebrae most commonly broken are those in the lower back. FIG. 1 is an illustration showing an example of a vertebral compression fracture. In this example, surgery may be required to prevent the spine from pressing on the spinal cord or to stabilize the vertebra adjacent to the fracture site. As proper exposure to the fracture site is necessary, patients undergoing such an invasive operation are at risk for infections as well as prolonged immobilization. A less invasive procedure, percutaneous vertebroplasty, is currently available for relieving chronic pain associated with osteoporotic fractures. In this procedure, a biopsy needle (e.g., a Jamshidi® needle) is inserted through a patient's skin and medical grade bone cement, poly methyl methacrylate (PMMA), is injected into the vertebra with the fracture. Other treatments may include steps to restore a natural height of a fractured vertebra followed by injection of bone cement or other material into a cavity in the vertebra. For example, a surgeon may use an inflatable balloon to compact bone, and then inject a biomaterial into a cavity. Such balloon procedures have been referred to as “kyphoplasty,” and equipment for such procedures may be available from Kyphon, Inc.

Limitations of such methods of cement introduction may include retrograde flowback of bone cement along the needle and limited vertebral body fill. Local complications from bone cement leakage (i.e., retrograde flowback) may include radiculopathy and cord compression. Systemic complications from bone cement leakage may include fever, infection, pulmonary embolism, fat embolism, hypoxia, hypotension, myocardial infarction, and sudden death. These limitations and potentially deadly complications demonstrate the risks and severe disadvantages of using the conventional bone cement to treat a vertebral compression fracture. One of the risks is the thermal damage to living tissue when the bone cement cures. The curing reaction of the conventional bone cement is exothermic and generates temperatures in the range of 80° C.-124° C., which is at least 50% higher than the limit for thermal damage to living tissue (48° C.-60° C.). In the case of PMMA, the polymerization process can lead to temperature rises sufficient to cause protein denaturation, cell necrosis and nerve ablation.

Equally important, the conventional bone cement and the natural (cancellous) bone have a severe mismatch problem in physical properties (e.g., stiffness) The modulus of a natural cancellous bone is 100 MPa-500 MPa while that of the conventional bone cement is 10 GPa-12 GPa. The stiffness mismatch between the conventional implanted material (e.g., the PMMA) and the surrounding cancellous bone is at least three orders of magnitude, causing adjacent vertebra stress transfer as well as undue stresses in operated vertebra. Thus, the stiffness mismatch can create an increased risk of facture to adjacent and operated vertebral bodies.

Another harmful side effect of using the conventional bone cement is the toxic monomer(s) released inside the human body during surgery. As an example, components of an acrylic bone cement may include, in powder form, a polymer (Polymethyimethacrylate/copolymer (PMMA)), an initiator (Benzoyl peroxide (BPO)), a radio-opacifer (Zirconium dioxide (ZrO₂)), antibiotics (gentamicin, etc.) and, in liquid form, a monomer (Methylmethacrylate (MMA)), an accelerator (N,N-Dimethyl para-toluidine (DMPT)), and a stabilizer (hydroquinone, etc.). Toxic substances released/leaked into the body when PMMA cures may include monomers such as MMA and DMPT. These toxic substances can cause severe blood pressure drop, increasing the risk of complication. More specifically, when a surgeon presses the doughy bone cement into the prepared cavity in the bone, small quantities of the monomer fluid are still present in the product. The toxic monomer fluid may leak into the circulation and cause the sudden blood pressure fall during the operation.

The conventional bone cement itself is difficult to handle. Once prepared, the chemical reaction of curing begins almost immediately, which leaves a very small window of time for finding the precise location and injecting the right amount of the conventional bone cement to where it needs to be. As the conventional bone cement cures, it is very difficult, and would be very messy, to make an adjustment.

### SUMMARY OF THE INVENTION

Embodiments of the present invention provide a new solution to repair a vertebral compression fracture (VCF). The solution includes a unique biocompatible substance and methods of making and deploying the biocompatible substance in the vicinity of VCF in order to heal the fracture The biocompatible substance can be injected in liquid form at room temperature and cure at body temperature inside a body. The biocompatible substance according to the invention has optimum mechanical properties that mimic natural bone and presents a new approach to treating vertebral compression fractures without harmful side effects. The biocompatible substance can be used alone in minimally invasive procedures or in combination with other spine treatments such as medication or implants (e.g., nucleus replacement).

More specifically, embodiments of the invention provide an injectable co-polymer hydrogel made from at least two biocompatible polymeric hydrogels as its constituents. The hydrogels are from a class of polymers which undergo volume and stage changes with temperature. More specifically, the hydrogels or combinations thereof exhibit a lower critical solution temperature (LCST) phenomenon. The materials of this class are in liquid form below their LCST and convert from liquid to solid above LCST. Particularly, the materials which exhibit LCST in the range of 25° C.-34° C. are of special interest. Because of the inherent characteristics of LCST, such a material would be in liquid form at room temperature (e.g., 25° C.) prior to and during deployment and would convert to solid form within the human body (37° C.) after deployment. That is, the biocompatible substance of the present invention will not begin to harden until after it is in place (i.e., injected or otherwise deployed to the fracture site). In one embodiment, the hardened biocompatible substance has stiffness properties manipulated to match with the human cancellous bone.

Embodiments of the invention can be made from two or more such polymeric materials via physical cross-linking Physically cross-linking manufacturing methods do not require cross-linking agents and produce no leachable substances once inside the human body. To this extent, physical cross-linking has advantages over chemical cross-linking Chemical cross-linking is a highly versatile method to create hydrogels with good mechanical stability However, chemical cross-linking agents used in preparing such hydrogels are often toxic compounds, which have to be extracted or otherwise removed from the hydrogels before they can be applied. Moreover, chemical cross-linking agents can give unwanted reactions with the bioactive substances present in the hydrogels. Without using chemical cross-linking agents, physically cross-linked hydrogels can advantageously avoid these adverse effects.

Additionally, this invention provides a number of advantages, including but not limited to the following. The biocompatible substance according to the invention can be used in minimally invasive procedures such as percutaneous vertebroplasty, reducing the risk of complication and the length of immobilization. Moreover, the mechanical properties of the biocompatible substance according to the invention can be easily tuned or otherwise manipulated so as to match with that of cancellous bone modulus, reducing or eliminating the risk of adjacent vertebra stress transfer. Furthermore, the biocompatible substance according to the invention can cure at body temperature and thus would not cause thermal damage to living tissues Another advantage is that the biocompatible substance according to the invention does not release toxic monomers and thus avoids the harmful side effect of causing severe blood pressure drop during the procedure. Yet another advantage is that the composition of the biocompatible substance according to the invention can be tailored for different uses (e.g., as a drug carrier, growth factor carrier, etc.) for vertebral cancer treatments or some other treatments as desired.

Other objects and advantages of the invention will be better appreciated and understood when considered in conjunction with the following description and the accompanying drawings.

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein:

FIG. 1 is an illustration showing an exemplary side view of a vertebral compression fracture site;

FIG. 2 is an illustration showing an exemplary top view of a vertebral body and various approaches to delivering a biocompatible substance for repairing a bone, according to some embodiments of the invention.

FIG. 3 is an illustration representative of a minimally invasive method of delivering a biocompatible substance for repairing a damaged or diseased bone using a cannulated device such as a syringe, according to one embodiment of the invention.

FIG. 4 is an illustration representative of another minimally invasive method of delivering a biocompatible substance for repairing a damaged or diseased bone using a cannulated device such as a bone tap, according to one embodiment of the invention;

FIGS. 5A-E are illustrations representative of another minimally invasive method of delivering a biocompatible substance for repairing a damaged or diseased bone using a guide wire, a dilator, a cannulated device such as a bone tap, and a driver, according to one embodiment of the invention; and

FIG. 6 is an illustration showing an exemplary top view of a vertebral body repaired with a biocompatible substance after a vertebroplasty procedure, according to some embodiments of the invention.

### DETAILED DESCRIPTION OF THE INVENTION

The invention and the various features and advantageous details thereof are explained more fully with reference to the non-limiting embodiments detailed in the following description Descriptions of well known starting materials, manufacturing techniques, components and equipment are omitted so as not to unnecessarily obscure the invention in detail. Skilled artisans should understand, however, that the detailed description and the specific examples, while disclosing preferred embodiments of the invention, are given by way of illustration only and not by way of limitation Various substitutions, modifications, and additions within the scope of the underlying inventive concept(s) will become apparent to those skilled in the art after reading this disclosure Skilled artisans can also appreciate that the drawings disclosed herein are not necessarily drawn to scale.

Embodiments of the present invention provide a biocompatible substance useful for repairing a vertebral compression fracture (VCF). The biocompatible substance can be injected or otherwise deployed in the vicinity of a VCF in order to heal the fracture. The biocompatible substance according to the invention presents a new approach to treating the VCF. Unlike the conventional bone cement (PMMA), the biocompatible substance according to the invention has no known harmful side effects.

The biocompatible substance according to the invention includes an injectable copolymer hydrogel made from at least two biocompatible polymeric hydrogels as its constituents and having optimally manipulated physical properties comparable to those of a natural bone (e.g., the human cancellous bone) Hydrogels are polymeric networks, which absorb and retain large amounts of water. In such a polymeric network, hydrophilic groups or domains are present which are hydrated in an aqueous environment Cross-links in the polymeric network prevent dissolution of the hydrophilic polymer chains or segments into the aqueous phase. The hydrogels of the present invention are from a class of polymers which undergo volume and state changes with temperature. Specifically, these hydrogels or combinations thereof exhibit a lower critical solution temperature (LCST) phenomenon. The LCST phenomenon refers to the fact that the miscibility of two non-identical polymers decreases with increasing temperature. In embodiments of the invention, a biocompatible substance is a polymer mixture made of two different polymeric components, has LCST behavior and displays temperature dependent light permeability. At least one of the polymeric components also exhibits LCST. Examples of suitable hydrophilic polymers which exhibit LCST include, but not limited to, N-isopropylacrylamide (NIPAAm) hydrogels and co-polymers, including pNIPAAM/poly(ethylene glycol) (PEG), pNIPAAM/CMCS (carboxymethyl chitosan), pNIPAAM supported on alginate-Ca2+, etc., poly(N,N-diethylacrylamide), poly(vinyl methyl ether), glycerin-polypropylenglycol-ether (GP), glycerin-polyethylenglycol-ether (GEP), etc. Some of these polymeric components are commercially available in various grades and qualities. Additional representative examples of polymer mixtures with LCST behavior can be found in the U.S. Pat. No. 5,430,104, which is incorporated herein by reference.

Embodiments of the invention can be made from two or more such polymeric materials as follows:
Purify the polymer with inert solvents (e.g., methanol, hexane, etch);
Perform free radical polymerization of oligomers using inert gases (e.g., nitrogen, argon, etc.) and suitable solvents (e.g., acetone, methanol, water etc.) in the presence of initiators (e.g., ammonium persulfate AmPS, sodium metabisulfite NaMBS, azobisisobutyronitrile AIBN) and/or cross-linking agents (e.g., methylenebisacrylamide MBAAm) and/or chain transfer agents (e.g., 2-amino ethanethiol hydrochloride AET.HCL) and/or reinforcing agents and/or bonding agents;
Perform the polymerization for a predetermined/desired time at or above room temperature;
Remove solvent traces using standard cleansing and drying methods (e.g., precipitation, vacuum, heat, etc.); and
Prepare the polymer solution with solvent, if needed.

In one embodiment, the polymer solution (i.e., a biocompatible substance according to one embodiment of the present invention) thus made does not produce leachable toxins inside the human body. There is no monomer release and the reaction is not exothermic. As mentioned before, the conventional bone cement has a high probability of leachable substances and subsequent undesirable side effects, including severe blood pressure drop during the surgery. Thus, the biocompatible substance of the invention can reduce complications commonly caused by using conventional bone cement. Another advantage of the biocompatible substance of the invention is that its strength can be tailored to match needs and applications More specifically, in embodiments of the invention, the branching of the polymers can be varied to control the physical strength and compressibility of the hydrogels thus made (i.e., the greater the degree of branching and the shorter the branches, the greater the strength of the hydrogels, and the smaller the pores, the lower the water content.) Strength in this context is defined as resistance to compression or stretching.

The LCST phenomenon refers to the temperature-sensitive phase transition of the polymer network. Many polymer solutions exhibit LCST, under which the polymer is soluble and above which the polymer is insoluble, and solidifies in a few minutes. The transition from a homogeneous mixture to phase separation can occur within 1-2° C. The polymeric materials in embodiments of the invention are in liquid form below their LCST and convert from liquid to solid while above LCST. In some embodiments, the polymeric materials implementing the present invention exhibit LCST in the range of 25° C.-34° C. As an example, the LCST of N-isopropylacrylamide (NIPAAm) is near ambient temperatures. Because of the inherent characteristics of the LCST, such materials will be liquid at room temperature (e.g., 25° C.) and would convert to solid form within the human body (37° C.). For further discussions on the LCST phenomenon, readers are directed to “Lower Critical Solution Temperature Behavior in Polymer Blends: Compressibility and Directional-Specific Interactions,” Macromolecules 1984, 17, 815-820, by Gerrit ten Brinke and Frank E. Karasz, the content of which is incorporated herein by reference.

As the biocompatible substance according to the invention can cure at body temperature, it would not cause thermal damage to living tissues. Furthermore, since the biocompatible substance of the present invention can remain in liquid form at room temperature prior to being injected or otherwise deployed to the fracture site, it can shorten the delivery time and lessen the effort to correct or make any necessary adjustments during the procedure.

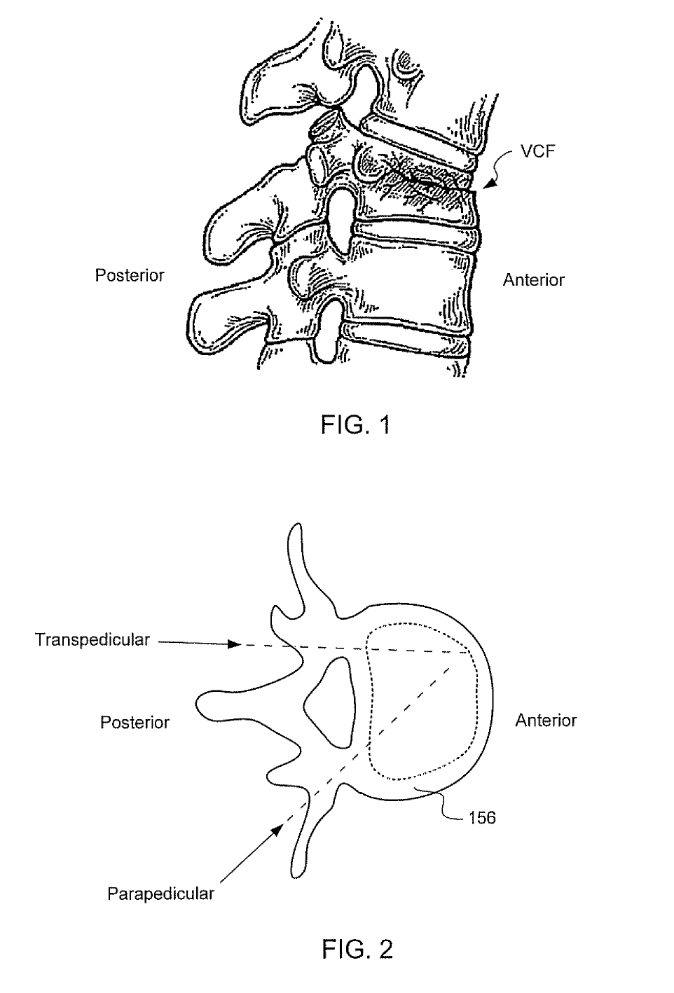

FIG. 2 is an illustration showing different ways of delivering a biocompatible substance according to the invention to a desired place for repairing a damaged or diseased bone such as the vertebral compression fracture site of FIG. 1. As an example, the biocompatible substance can be injected in the vicinity of the compression fracture in vertebroplasty procedure using a posterior approach. Either a transpedicular route or a parapedicular route can be used. Alternatively, an intercostal approach through the ribs (not shown) or a more invasive anterior route may be used.

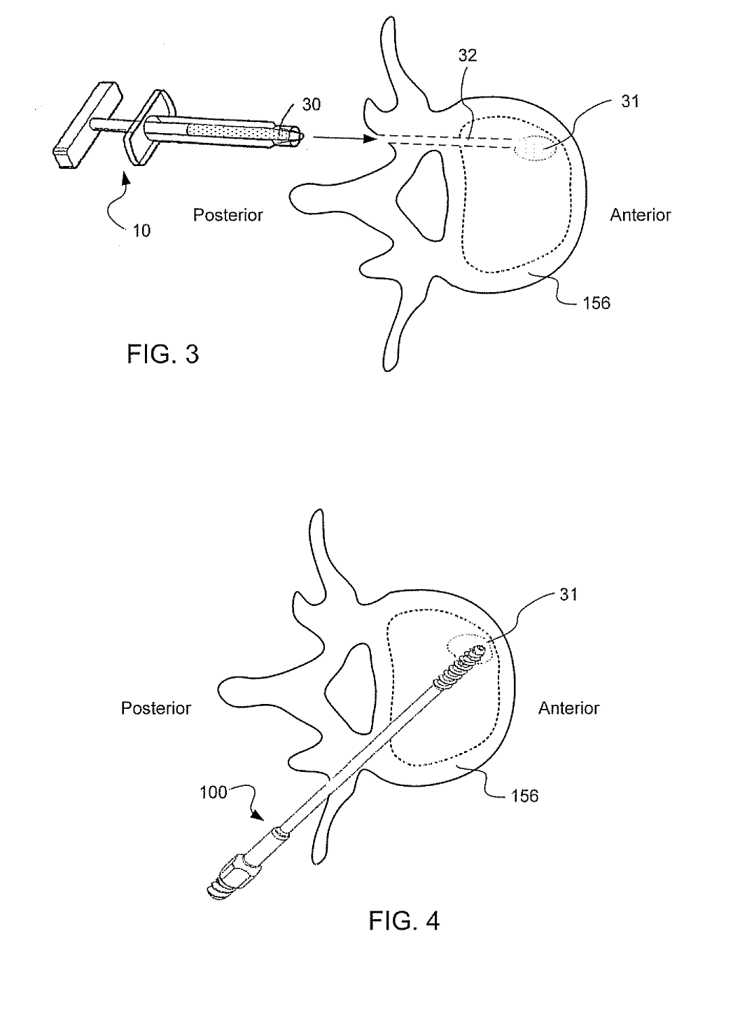

FIG. 3 is an illustration representative of a minimally invasive method of delivering a biocompatible substance for repairing a damaged or diseased bone, according to one embodiment of the invention In one embodiment, a transpedicular approach is used in a vertebroplasty procedure In one embodiment, the vertebroplasty procedure uses a cannulated device In one embodiment, the cannulated device is syringe 10 containing biocompatible substance 30 for filling site 31 in bone 156. In one embodiment, site 31 may comprise a cavity. In one embodiment, site 31 may further comprise cracks or channels. In one embodiment, the amount of biocompatible substance 30 is sufficient to fill site 31 in bone 156 In one embodiment, the amount of biocompatible substance 30 is sufficient to fill site 31 in bone 156 and conduit 32.

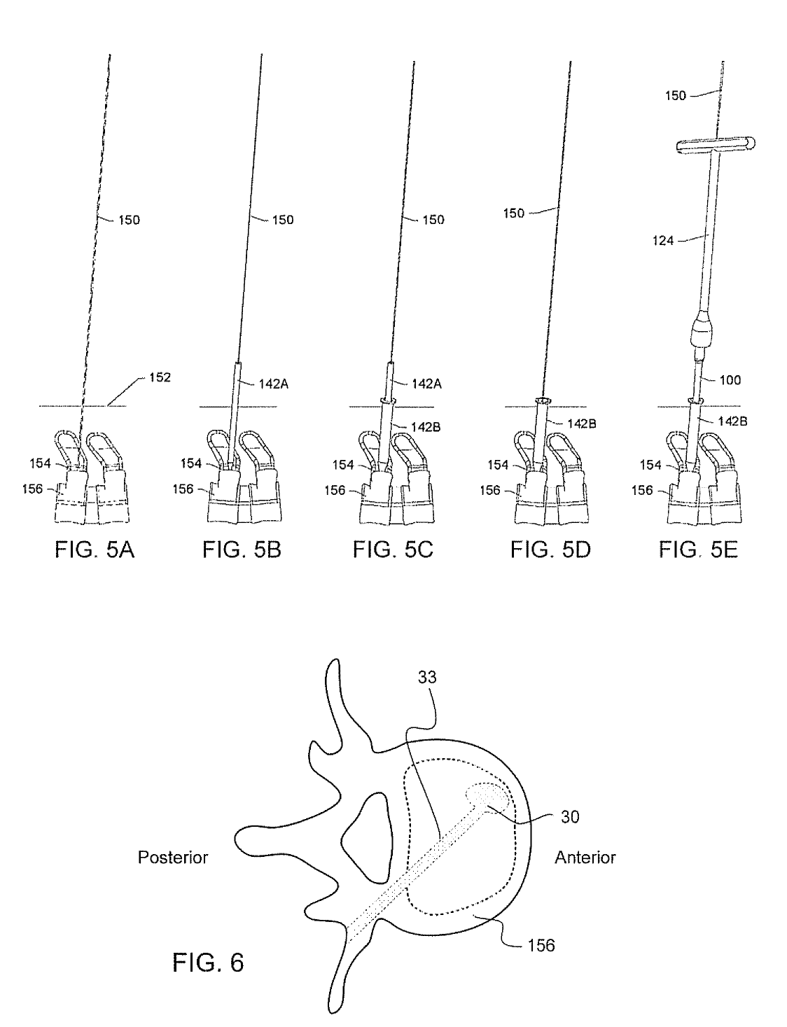

In some embodiments, a fenestrated bone tap may be used in a procedure to introduce the biocompatible substance into a fractured or diseased bone In some embodiments, the bone may be a vertebra For example, vertebroplasty may be used to treat a compression fracture resulting from osteoporosis in a vertebra. The vertebra may include, but is not limited to, a lumbar vertebra or a lower thoracic vertebra. In some embodiments, a fenestrated bone tap may be used to introduce the biocompatible substance into a void in a bone created by surgical removal of a tumor. Bone taps used for vertebroplasty may be similar to bone tap 100 depicted in FIGS. 4-5.

FIG. 4 is an illustration representative of another minimally invasive method of delivering a biocompatible substance for repairing a damaged or diseased bone, according to one embodiment of the invention. In one embodiment, a parapedicular approach is used in a vertebroplasty procedure. In one embodiment, the vertebroplasty procedure uses a cannulated device. In one embodiment, the cannulated device is bone tap 100 having a reservoir containing biocompatible substance 30 for filling site 31 in bone 156. In one embodiment, site 31 may comprise a cavity. In one embodiment, site 31 may further comprise cracks or channels. In one embodiment, the amount of biocompatible substance 30 is sufficient to fill site 31 in bone 156 In one embodiment, the amount of biocompatible substance 30 is sufficient to fill site 31 in bone 156 and conduit 33.

FIGS. 5A-E are illustrations representative of another minimally invasive method of delivering a biocompatible substance for repairing a damaged or diseased bone using a guide wire, a dilator, a cannulated device such as a bone tap, and a driver, according to one embodiment of the invention. More specifically, FIG. 5A depicts guide wire 150 positioned through an incision formed in skin 152. Guide wire 150 is inserted into pedicle 154 and vertebral body 156.

Guide wire 150 may be used as a guide to position one or more successively sized dilators (e.g., dilators 142A, 142B) at a target location. A dilator may form an opening through soft tissue to vertebral body 156 For patients with a thick fascia, it may be advantageous to make a nick in the fascia with a scalpel blade to facilitate passage of dilators 142A, 142B. Dilators 142A, 142B may be passed sequentially over guide wire 50, as illustrated in FIGS. 5B and 5C. Dilators 142A, 142B may be rotated during insertion to facilitate dilation of surrounding tissue. Once second dilator 142B is in position, first dilator 142A may be removed, as illustrated in FIG. 5D. Lengths of dilators in a successively sized set may decrease with increasing diameter to facilitate removal of the smaller dilators. Care should be taken to avoid dislodging guide wire 150 during insertion and removal of the dilators Additional dilator(s) may be inserted until the leading edges contact vertebral body 156. A distal end of a dilator may be tapered to facilitate positioning of the dilator proximate vertebral body 156.

After tissue dilation has been achieved, guide wire 150 and a large diameter dilator (e.g., second dilator 142B shown in FIG. 5D) may be used to guide a bone tap and/or bone fastener insertion instruments toward a target location. In some embodiments, a bone awl may be used to breach vertebral bone to allow for insertion of the bone tap. In some embodiments, an initial passage may be formed in pedicle 154 and vertebral body 156 using a drill. FIG. 5E depicts bone tap 100 positioned in second dilator 142B. Bone tap 100 may be sized to fit snugly inside second dilator 142B. Bone tap 100 may be coupled to driver 124. In one embodiment, bone tap 100 is a fenestrated bone tap.

Biocompatible substance 30 may be introduced through bone tap 100 as it is being withdrawn from bone or vertebra 156 to fill a central passage formed by bone tap 100 and to provide a long stabilization area in bone 156. If needed, a wrench coupled to a tool portion of bone tap 100 may be used to facilitate backout of bone tap 100 so that biocompatible substance 30 may be introduced into the long stabilization area, one example of which is illustrated in FIG. 6.

In some embodiments, one or more bones adjacent to a target bone may be augmented before a target bone is augmented For example, a vertebroplasty may be performed to correct a fractured L4 vertebra. Before tapping and injecting a biocompatible substance of the invention into the target L4 vertebra, a surgeon may tap and inject bone cement into the L3 vertebra (immediately superior to the L4 vertebra) and the L5 vertebra (immediately inferior to the L4 vertebra). The biocompatible substance of the invention may address weakness in the L3 and L5 vertebrae caused by osteoporosis or other factors After the biocompatible substance of the invention in the L3 and L5 vertebra has partially or fully cured, the surgeon may tap and inject a desired amount of the biocompatible substance of the invention into the target L4 vertebra. Using bone augmentation to strengthen bones adjacent to a target bone may reduce the risk of a strengthened target bone damaging a softer or weaker adjacent bone. In some embodiments, adjacent bones may be augmented with a biocompatible substance of the invention during or after augmentation of the target bone. In some embodiments, adjacent portions of a target portion of a single bone (e.g., a femur) may be augmented before, during, or after the target portion is augmented. Further modifications and alternative embodiments of various aspects of the invention will be apparent to those skilled in the art in view of this description.

Moreover, those skilled in the art can appreciate that many past and future cannulated devices can be used to deliver embodiments of the biocompatible substance to a target location of a damaged or diseased bone in a similar manner as described above. In addition to syringes and bone taps, cannulae, tubes, hollowed rods, catheters, cannulated screws, intraosseous needles, and the like may also be used. Additional exemplary cannulated devices are described in U.S. Pat. Nos. 6,960,215, 6,613,018, 5,484,442, 4903,691, 4,537,185, and U.S. Patent Application Publication Nos. US 2006/0229628, US 2004/0092946, US 2003/0212426, and US 2002/0120240, the contents of which are incorporated herein by reference. Accordingly, cannulated devices shown in the figures of the present invention are meant to be exemplary and by no means limiting.

FIG. 6 is an illustration showing an exemplary top view of a vertebral body repaired with a biocompatible substance after a vertebroplasty procedure, according to some embodiments of the invention. As illustrated in FIG. 6, upon injection, the liquid form of biocompatible substance 30 fills site 31 in the damaged bone 156 and begins to cure within the human body. In one embodiment, biocompatible substance 30 also fills passage 33. In one embodiment, to increase the bonding to bone, bioactive material(s) such as bonding agent(s) and/or fibrosis-inducing agent(s) may be added to biocompatible substance 30 during the aforementioned free radical polymerization.

The biocompatible substance according to the invention can cure within the human body without damaging living tissues. As an example, one embodiment of the biocompatible substance can cure in the human body within three to five minutes after injection to fill the cavity, crack(s) or fracture profile in the damaged vertebral body and convert into solid form. As described below, in the cured state, one embodiment of the biocompatible substance according to the invention has a stiffness that matches or is comparable to that of human cancellous bones.

As described herein, the stiffness and strength of the biocompatible substance can be easily manipulated in several ways (e.g., by controlling the branching of one or more of the polymeric components, by introducing one or more suitable reinforcing agents, etc.) so as to match with that of cancellous bone modulus, reducing or eliminating the risk of adjacent vertebra stress transfer In one embodiment, the modulus of the biocompatible substance according to the invention is in the range of 200 MPa-500 MPa. The modulus of natural cancellous bone is in the range of 100 MPa-500 MPa. The manipulation of the biocompatible substance can be done by adding an optimum amount (e g., 10-20% by volume) of reinforcement such as nano-HA particles, ceramic whiskers, UHMWPE globules or carbon nanotubes. The increase in the stiffness of the biocompatible substance can correspond to the amount of reinforcing substance present. As the mechanical efficacy of vertebroplasty can depend on bone mineral density (BMD), fracture severity, and disc degeneration, the ability of the biocompatible substance to restore the spine's mechanical properties following fracture can be optimized in various ways (e.g., through empirical techniques). Further discussions on the mechanical efficacy of vertebroplasty can be found in “Mechanical Efficacy of Vertebroplasty: Influence of Cement Type, BMD, Fracture Severity, and Disc Degeneration,” Bone 40 (2007) 1110-1119, by Jin Luo et al., the content of which is incorporated herein by reference.

For the sake of surgery, the biocompatible substance according to the invention can be radiopaque with the addition of a radiopaque agent(s) such as BaSO₄. Depending on the situation, the composition of the biocompatible substance according to the invention can be tailored to use it as drug carrier for vertebral cancer treatments or some other treatments as desired. For example, it can be used in conjunction with medication or implants, including total disc replacement, nucleus replacement or annulus repair devices.

Furthermore, embodiments of the biocompatible substance disclosed herein can be used alone or in combination with other devices and/or spine treatments in minimally invasive procedures such as vertebroplasty, reducing the risk of complication and the length of immobilization. In some embodiments, standard surgical instruments such as bone biopsy syringes may be used in a procedure (e.g., percutaneous vertebroplasty, kyphoplasty, etc.) to introduce the biocompatible substance into a fractured or diseased bone. In this way, no special delivery device and method would be necessary, advantageously minimizing the cost involved in adapting the technology.

As another example, in one embodiment, the sequence of a minimally invasive operation for deployment of the biocompatible substance according to the invention can comprise the following. First, materials are prepared. This may include an injectable copolymer hydrogel according to the invention, 11- or 13-gauge bone biopsy needles with connection tubing, and injection syringes. Skilled artisan can appreciate that injection syringes are only one of many ways to inject the biocompatible substance of the invention As mentioned above, many commercial kits for vertebroplasty and kyphoplasty intervention procedures can be utilized The procedure should be performed in a sterile operating environment and all materials should be sterilized In one embodiment, the injectable copolymer hydrogel according to the invention is transferred from a container into a sterilized pressure syringe. The amount of the injectable copolymer hydrogel in the syringe may vary, depending upon the VCF that needs to be treated Local anesthesia is then applied. This can be done using a local anesthetic solution as known to clinicians trained to perform vertebroplasty and/or kyphoplasty intervention procedures. In one embodiment, additional anesthesia (e g., general anesthesia, monitored anesthesia, etc.) may be performed if necessary.

A surgeon or a trained clinician may next use a bone biopsy needle to puncture a patient's skin and perform a vertebral body biopsy and/or venography. Other types of minimally invasive incisions may also be applied to gain access to the VCF site. Venography may provide anatomical knowledge of the large venous channels in proximity to the VCF site, enabling the clinician to inject the liquid copolymer hydrogel with more care. This modality can be optional. In some embodiments, other imaging modalities can be used to facilitate the operation. After confirming the injection site, the surgeon or clinician can then carefully inject the liquid copolymer hydrogel to fill the cracks. In one embodiment, the injection pressure should be at least 0.3 MPa (i.e., about 43.51 psi or more). A person with a 10 cc syringe can easily generate pressures of about 100-150 psi. Since the procedure generally uses about 3.5 cc of implant material per side of the vertebral body, the relatively low pressure requirement allows the clinician to deliver a desired amount of the substance in a single “shot”, using only one syringe. With conventional bone cements such as PMMA, the clinician can only deliver a small amount of viscous material at a time, as higher pressures are required to deliver conventional bone cements and smaller syringes (erg., 1 cc) can achieve higher pressures (erg., 800-1200 psi). Another way to deliver PMMA is to use a specially designed system that provides pressures up to 4000 psi, an example of which is disclosed in U.S. Patent Application Publication No. US 2006/0266372 A₁, entitled “HIGH PRESSURE DELIVERY SYSTEM,” filed Mar. 6, 2006.

In one embodiment, the solidness of the biocompatible substance can be manipulated during the procedure by subjecting the biocompatible substance to an environment (e.g., a warm water bath) having a temperature slightly above or around the LCST of the biocompatible substance. In some embodiments, a semi-solidified biocompatible substance can be injected to confine a liquid biocompatible substance from spreading beyond the operation site (e.g., through a venous channel).

Embodiments of the invention serve to fill the cracks in damaged vertebra, thereby restoring the integrity of the vertebral body. With proper deployment, the biocompatible substance according to the invention can add stabilization to the spinal column and assist in proper load transfer. Embodiments of the invention also serve as a superior alternative to the current treatments of VCF which use conventional bone cement exhibiting harmful side effects. According to one embodiment of the invention, the biocompatible substance can be easily manufactured from common and inexpensive hydrogels, such as those biocompatible polymers listed above.

In the foregoing specification, the invention has been described with reference to specific embodiments However, as one skilled in the art can appreciate, embodiments of the invention disclosed herein can be modified or otherwise implemented in many ways without departing from the spirit and scope of the invention. Accordingly, this description is to be construed as illustrative only and is for the purpose of teaching those skilled in the art the manner of carrying out the invention. It is to be understood that the forms of the invention herein shown and described are to be taken as exemplary embodiments. Equivalent elements or materials may be substituted for those illustrated and described herein. Moreover, certain features of the invention may be utilized independently of the use of other features, all as would be apparent to one skilled in the art after having the benefit of this description of the invention.

What is claimed is:

1. A biocompatible substance useful for repairing a bone fracture, comprising:
two or more polymeric components, at least one of which exhibits a lower critical solution temperature (LCST) behavior;
wherein the two or more polymeric components are selected from a group consisting of N-isopropylacrylamide (NIPAAm), pNIPAAM/poly(ethylene glycol) (PEG), pNiPAAMICMCS (carboxymethyl chitosan), pNIPMM supported on alginate-Ca2+, poly(N,N-diethylacrylamide), poly(vinyl methyl ether), glycerin-polypropylenglycol-ether (GP), and glycerin-polyethylenglycol-ether (GEP);
wherein the biocompatible substance is injectable at or near room temperature;
wherein the biocompatible substance cures in a human body at or near body temperature;
wherein the cured biocompatible substance has a modulus in the range of about 200 MPa to about 500 MPa; and
wherein the modulus of the cured biocompatible substance matches or is comparable to that of the bone.

2. The biocompatible substance of claim 1, wherein, when in an environment having a temperature above the LCST, the biocompatible substance converts to solid form in about three to five minutes.

3. The biocompatible substance of claim 1, further comprising a reinforcement agent.

4. The biocompatible substance of claim 3, wherein the reinforcement agent comprises nano-HA particles, ceramic whiskers, UHMWPE globules, carbon nanotubes, or a combination thereof.

5. The biocompatible substance of claim 1, further comprising at least one radiopaque agent.

6. The biocompatible substance of claim 1, wherein at least one of the two or more polymeric components undergo volume and state changes with temperature.

7. The biocompatible substance of claim 1, wherein at least one of the two or more polymeric components exhibit the LCST from about 25° C to about 34° C.

8. The biocompatible substance of claim 1, wherein when in an environment having a temperature below the LCST, the biocompatible substance is injectable under a pressure of about 0.3 MPa or more.

9. A method for delivering a biocompatible substance for repairing a bone fracture, comprising:
making a minimally invasive incision to gain access to the bone fracture;
inserting a cannulated device through the minimally invasive incision; and
flowing the biocompatible substance to the bone fracture through the cannulated device under a pressure of about 0.3 MPa or more;
wherein the biocompatible substance comprises two or more polymeric components, at least one of which exhibits a lower critical solution temperature (LCST) behavior;
wherein the biocompatible substance converts to solid form in a human body at or near body temperature;
wherein the biocompatible substance in solid form has a modulus in the range of about 200 MPa to about 500 MPa; and
wherein the modulus of the biocompatible substance in solid form matches or is comparable to that of the bone.

10. The method of claim 9, further comprising injecting the biocompatible substance at or near room temperature.

11. The method of claim 9, further comprising dilating the minimally invasive incision.

12. The method of claim 9, wherein the biocompatible substance further comprises a reinforcement agent.

13. The method of claim 12, wherein the reinforcement agent comprises nano-HA particles, ceramic whiskers, UHMWPE globules, carbon nanotubes, or a combination thereof.

14. The method of claim 9, wherein the biocompatible substance further comprises at least one radiopaque agent.

15. The method of claim 9 wherein the biocompatible substance further comprises at least one bioactive agent that facilitates bond bonding.
